(19) United States
(12) Patent Application Publication
(10) Pub. No.: US 2024/0325272 A1
(43) Pub. Date: Oct. 3, 2024

(54) PERSONAL CARE COMPOSITIONS

(71) Applicant: COLGATE-PALMOLIVE COMPANY, New York, NY (US)

(72) Inventors: Dariusz DZIEKONSKI, Lawrence Township, NJ (US); Tochukwu OFOEGBUNA, New Brunswick, NJ (US); Hrebesh MOLLY SUBHASH, Somerset, NJ (US); Shyamala PILLAI, Hillsborough, NJ (US)

(73) Assignee: COLGATE-PALMOLIVE COMPANY, New York, NY (US)

(21) Appl. No.: 18/621,369

(22) Filed: Mar. 29, 2024

Related U.S. Application Data
(60) Provisional application No. 63/493,448, filed on Mar. 31, 2023.

(51) Int. Cl. A61K 8/60; A61K 8/31; A61K 8/34; A61K 8/37; A61K 8/73; A61Q 15/00

(52) U.S. Cl. CPC A61K 8/602; A61K 8/31; A61K 8/345; A61K 8/375; A61K 8/73; A61Q 15/00; A61K 2800/30; A61K 2800/48

(57) ABSTRACT

Described herein are personal care compositions comprising a cosmetically acceptable carrier; an antiperspirant component comprising gluconolactone, or an acid thereof; and optionally a thickening agent. Methods of making and using these compositions are also described herein.

### CROSS-REFERENCE TO RELATED APPLICATIONS

This application claims the benefit of priority from U.S. Patent Application No. 63/493,448, filed Mar. 31, 2023, titled Personal Care Compositions, the content of which is hereby incorporated herein in its entirety, for all purposes.

### BACKGROUND

Personal care compositions, such as antiperspirant or deodorant compositions may be used to reduce body odor. Antiperspirant or deodorant compositions may be applied to axillary (underarm) regions to prevent or treat perspiration, limit the growth of odor-causing bacteria, or apply a fragrance. Antiperspirant or deodorant compositions may be delivered topically as roll-on, gel, cream, stick or aerosol formulations.

Underarm deodorants and antiperspirants typically control odor by eliminating the bacteria that cause odor. Conventional antiperspirant salts, such as aluminum, and/or zirconium salts, tend to be acidic in aqueous solution, a property which makes them effective bactericides, thereby providing a deodorant benefit. In addition, long-acting antiperspirant compositions typically contain increased amounts of active ingredients as a route to obtaining sustained effectiveness. At the same time, skin sensitivity and vulnerability to various compounds may limit the practical upper concentration in personal care compositions.

Generally, aluminum salts are the active ingredients of antiperspirant products. In recent years there has been increasing consumer pressure to reduce or remove aluminum salts from antiperspirant or deodorant type products. However, replacing aluminum salts in antiperspirant and deodorant products, but at the same time maintaining the same desirable sweat blocking properties consumers value in an antiperspirant or deodorant product, is a challenge within the personal care composition industry.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

Aspects of the present invention are directed to personal care composition that may be in the form of a deodorant and/or an antiperspirant. In accordance with an aspect of the invention, provided is a personal care composition comprising a cosmetically acceptable carrier; an antiperspirant component comprising gluconolactone, and/or an acid thereof; and optionally a thickening agent. In some embodiments, the antiperspirant component further comprises an ingredient selected from: lactobionic acid, maltobionic acid, galactonolactone, glucuronolactone, galacturonolactone, gulonolactone, ribonolactone, saccharic acid lactone, pantolactone, glucoheptonolactone, mannonolactone, galactoheptonolactone, an acid thereof, and a salt thereof, or a combination of two or more thereof. In accordance with another aspect, provided is a personal care composition comprising an antiperspirant component comprising gluconolactone and/or acids thereof; from about 0.1 to about 7 wt. % of a thickening agent; from about 0.1 to about 25 wt. % of a polyol; and water, wherein all weight percentages are based on the total weight of the personal care composition. In accordance with a further aspect of the invention, provided is a personal care composition comprising an antiperspirant component consisting essentially of gluconolactone and/or an acid thereof.

Further embodiments of the present invention provide personal care compositions wherein the antiperspirant component comprises gluconolactone, or an acid thereof; and a metal oxalate. In some embodiments, the metal oxalate is selected from potassium oxalate; potassium titanium oxide oxalate; and a combination thereof.

A list of non-limiting, example embodiments is provided below:

In accordance with an embodiment 1, provided is a personal care composition comprising:
a cosmetically acceptable carrier;
an antiperspirant component comprising gluconolactone; and
optionally, a thickening agent.

In accordance with an embodiment 2, provided is a personal care composition according to embodiment 1, wherein the antiperspirant component further comprises an ingredient selected from: maltobionic acid, lactobionic acid, galactonolactone, glucuronolactone, galacturonolactone, gulonolactone, ribonolactone, saccharic acid lactone, pantolactone, glucoheptonolactone, mannonolactone, galactoheptonolactone; and a combination of two or more thereof.

In accordance with an embodiment 3, provided is a personal care composition according to embodiment 1 or embodiment 2, further comprising an additional polyhydroxy acid and/or salt thereof.

In accordance with an embodiment 4, provided is a personal care composition according to embodiment 3, wherein the additional polyhydroxy acid and/or salt thereof comprises galactose, lactobionic acid, or combinations thereof.

In accordance with an embodiment 5, provided is a personal care composition according to embodiment 3 or embodiment 4, wherein the additional polyhydroxy acid and/or salt thereof is present in an amount from about 0.1 to about 7 wt. %, based on the total weight of the personal care composition.

In accordance with an embodiment 6, provided is a personal care composition according to any foregoing embodiment further comprising an alpha hydroxy acid and/or salt thereof.

In accordance with an embodiment 7, provided is a personal care composition according to embodiment 6, wherein the alpha hydroxy acid comprise glycolic acid, citric acid, lactic acid, malic acid, tartaric acid, phytic acid, hydroxycaprylic acid, hydroxycapric acid, a salt thereof, or a combination of two or more thereof.

In accordance with an embodiment 8, provided is a personal care composition according to embodiment 6 or embodiment 7, wherein the alpha hydroxy acid and/or salt thereof is present in an amount from about 0.1 to about 7 wt. %, based on the total weight of the personal care composition.

In accordance with an embodiment 9, provided is a personal care composition according to any foregoing embodiment further comprising from about 0.1 to about 15 wt. % of an emollient, based on the total weight of the personal care composition.

In accordance with an embodiment 10, provided is a personal care composition according to embodiment 9, wherein the emollient is selected from the group consisting of: isododecane, sodium starch octenylsuccinate, C₁₂₋₁₅ alkyl benzoate, mineral oil, caprylyl glycol, dimethicone, dicaprylyl ether, ethyl trisiloxane, PPG-14 butyl ether, PPG-3 myristyl ether, stearyl alcohol, stearic acid and salts thereof, glyceryl monoricinoleate, isobutyl palmitate, isocetyl stearate, isocetyl stearate, sulphated tallow, oleyl alcohol, propylene glycol, isopropyl laurate, mink oil, sorbitan stearate, cetyl alcohol, hydrogenated castor oil, stearyl stearate, hydrogenated soy glycerides, isopropyl isostearate, hexyl laurate, dimethyl brassylate, decyl oleate, diisopropyl adipate, n-dibutyl sebacate, diisopropyl sebacate, 2-ethyl hexyl palmitate, isononyl isononanoate, isodecyl isononanoate, isotridecyl isononanoate, 2-ethyl hexyl palmitate, 2-ethyl hexyl stearate, Di-(2-ethyl hexyl) adipate), Di-(2-ethyl hexyl) succinate, isopropyl myristate, isopropyl palmitate, isopropyl stearate, octacosanol, butyl stearate, polyethylene glycols, oleic acid, triethylene glycol, lanolin, castor oil, acetylated lanolin alcohols, acetylated lanolin, petrolatum, isopropyl ester of lanolin, fatty acids, mineral oils, butyl myristate, isostearic acid, palmitic acid, PEG-8 distearate, PEG-23 oleyl ether, olelyl oleate, isopropyl linoleate, cetyl lactate, lauryl lactate, myristyl lactate, quaternised hydroxy alkyl, aminogluconate, vegetable oils, isodecyl oleate, isostearyl neopentanoate, myristyl myristate, oleyl ethoxy myristate, diglycol stearate, ethylene glycol monostearate, myristyl stearate, isopropyl lanolate, paraffin waxes, glycyrrhizic acid, hydrocyethyl stearate amide, and a combination of two or more thereof.

In accordance with an embodiment 11, provided is a personal care composition to any foregoing embodiment further comprising from about 0.1 to about 25 wt. % of a polyol, based on the total weight of the personal care composition.

In accordance with an embodiment 12, provided is a personal care composition according to embodiment 11, wherein the polyol comprises ethylene glycol, propylene glycol, butylene glycol, hexylene glycol, glycerin, diglycerin, diethylene glycol, and dipropylene glycol, or a combination of two more thereof.

In accordance with an embodiment 13, provided is a personal care composition according to any foregoing embodiment further comprising from about 0.5 to about 9 wt. % of a structuring agent, based on the total weight of the personal care composition.

In accordance with an embodiment 14, provided is a personal care composition according to any foregoing embodiment, wherein the thickening agent is selected from the group consisting of: xanthan gum, guar gum, biosaccharide gum, cellulose, acacia seneca gum, sclerotium gum, agarose, pectin, gellan gum, hyaluronic acid, and a combination of two or more thereof.

In accordance with an embodiment 15, provided is a personal care composition according to any foregoing embodiment further comprising from about 0.1 to about 7 wt. % of a preservative, based on the total weight of the personal care composition.

In accordance with an embodiment 16, provided is a personal care composition according to embodiment 15, wherein the preservative comprises caprylyl glycol, phenoxyethanol, butylated hydroxytoluene, ethylenediaminetetraacetic acid, ethylhexylglycerin, citric acid, benzoic acid, or a combination of two or more thereof.

In accordance with an embodiment 17, provided is a personal care composition comprising:
an antiperspirant component comprising gluconolactone and/or an acid thereof; and
from about 0.1 to about 7 wt. % of a thickening agent; and
from about 0.1 to about 25 wt. % of a polyol;
wherein all weight percentages are based on the total weight of the personal care composition.

In accordance with an embodiment 18, provided is a personal care composition according to embodiment 17 further comprising an additional polyhydroxy acid and/or salt thereof.

In accordance with an embodiment 19, provided is a personal care composition according to any foregoing embodiment further comprising an alpha hydroxy acid and/or salt thereof selected from the group consisting of: glycolic acid, citric acid, lactic acid, malic acid, tartaric acid, phytic acid, hydroxycaprylic acid, hydroxycapric acid, a salt thereof, and a combination of two or more thereof.

In accordance with an embodiment 20, provided is a personal care composition comprising:
an antiperspirant component consisting essentially of gluconolactone and/or an acid thereof.

In accordance with an embodiment 21, provided is a personal care composition according to any foregoing embodiment, wherein the antiperspirant component consists of gluconolactone and/or an acid thereof.

In accordance with an embodiment 22, provided is a personal care composition according to any foregoing embodiment, wherein the personal care composition is essentially free of aluminum salts.

In accordance with an embodiment 23, provided is a personal care composition according to any foregoing embodiment, wherein the personal care composition is essentially free of zinc-based antiperspirant actives.

In accordance with an embodiment 124, provided is a personal care composition according to any foregoing embodiment, wherein the personal care composition is essentially free of iron-based antiperspirant actives.

In accordance with an embodiment 25, provided is a personal care composition according to any foregoing embodiment, wherein the personal care composition is essentially free of zirconium-based antiperspirant actives.

In accordance with an embodiment 26, provided is a personal care composition according to any foregoing embodiment, wherein the personal care composition is essentially free of titanium-based antiperspirant actives.

In accordance with an embodiment 27, provided is a personal care composition according to any foregoing embodiment, wherein the personal care composition is essentially free of magnesium-based antiperspirant actives.

In accordance with an embodiment 28, provided is a personal care composition according to any one of embodiments 1 to 19 and 23 to 27, wherein the antiperspirant component consists essentially of gluconolactone, lactobionic acid, maltobionic acid, galactonolactone, glucuronolactone, galacturonolactone, gulonolactone, ribonolactone, saccharic acid lactone, pantolactone, glucoheptonolactone, mannonolactone, galactoheptonolactone, an acid thereof, a salt thereof, or a combination of two or more thereof.

In accordance with an embodiment 29, provided is a personal care composition according to any one of embodiments 1 to 19 and 23 to 27, wherein the antiperspirant component consists of gluconolactone, lactobionic acid, maltobionic acid, galactonolactone, glucuronolactone, galacturonolactone, gulonolactone, ribonolactone, saccharic acid lactone, pantolactone, glucoheptonolactone, mannonolactone, galactoheptonolactone, an acid thereof, a salt thereof, or a combination of two or more thereof.

In accordance with an embodiment 30, provided is a method for treating hyperhidrosis in a mammalian subject, comprising applying a personal care composition according to any foregoing embodiment to a skin surface of a mammalian subject in need thereof.

In an accordance with an embodiment 31, provided is a method for reducing or preventing sweat production in a mammalian subject, comprising: applying a personal care composition according to any one of embodiments 1 to 29, to a skin surface of a mammalian subject in need thereof.

### BRIEF DESCRIPTION OF THE DRAWING

FIG. 1 is a bar graph depicting the reduction in sweat achieved by a non-limiting, exemplary personal care composition in accordance with an aspect of the invention.

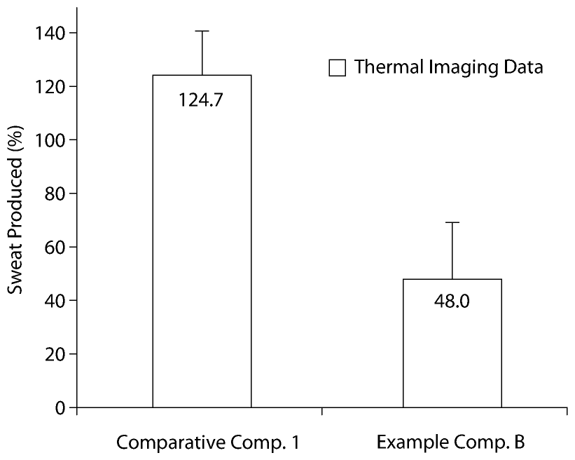

### DETAILED DESCRIPTION

For illustrative purposes, the principles of the present invention are described by referencing various exemplary embodiments thereof. Although certain embodiments of the invention are specifically described herein, one of ordinary skill in the art will readily recognize that the same principles are equally applicable to and can be employed in other compositions and methods. Before explaining the disclosed embodiments of the present invention in detail, it is to be understood that the invention is not limited in its application to the details of any particular embodiment disclosed herein. The terminology used herein is for the purpose of description and not of limitation.

As used herein and in the appended claims, the singular forms “a”, “an”, and “the” include plural references unless the context dictates otherwise. The singular form of any class of the ingredients refers not only to one chemical species within that class, but also to a mixture of those chemical species. The terms “a” (or “an”), “one or more” and “at least one” may be used interchangeably herein. The terms “comprising”, “including”, and “having” may be used interchangeably. The term “include” should be interpreted as “include, but are not limited to”. The term “including” should be interpreted as “including, but are not limited to”.

As used throughout, ranges are used as shorthand for describing each and every value that is within the range. Any value within the range can be selected as the terminus of the range. Thus, a range from 1-5, includes specifically 1, 2, 3, 4 and 5, as well as subranges such as 2-5, 3-5, 2-3, 2-4, 1-4, etc.

The term “about” when referring to a number means any number within a range of 10% of the number. For example, the phrase “about 2 wt. %” refers to a number between and including 1.8 wt. % and 2.2 wt. %.

All references cited herein are hereby incorporated by reference in their entireties. In the event of a conflict in a definition in the present disclosure and that of a cited reference, the present disclosure controls.

The abbreviations and symbols as used herein, unless indicated otherwise, take their ordinary meaning. The abbreviation “wt. %” means percent by weight with respect to the personal care composition. The symbol “°” refers to a degree, such as a temperature degree or a degree of an angle. The symbols “h”, “min”, “mL”, “nm”, and “μm” refer to hour, minute, milliliter, nanometer, and micrometer, respectively. The abbreviation “UV-VIS” referring to a spectrometer or spectroscopy, means Ultraviolet-Visible. The abbreviation “rpm” means revolutions per minute.

When referring to chemical structures, and names, the symbols “C”, “H”, and “O” mean carbon, hydrogen, and oxygen, respectively. The symbols “—”, “═” and “≡” mean single bond, double bond, and triple bond, respectively.

“Volatile”, as used herein, means having a flash point of less than about 100° C. “Non-volatile”, as used herein, means having a flash point of greater than about 100° C.

Any member in a list of species that are used to exemplify or define a genus, may be mutually different from, or overlapping with, or a subset of, or equivalent to, or nearly the same as, or identical to, any other member of the list of species. Further, unless explicitly stated, such as when reciting a Markush group, the list of species that define or exemplify the genus is open, and it is given that other species may exist that define or exemplify the genus just as well as, or better than, any other species listed.

The phrases, “a mixture thereof,” “a combination thereof,” or a combination of two or more thereof” do not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.” Likewise, the term “a salt thereof” also relates to “salts thereof.” Thus, where the disclosure refers to “an element selected from the group consisting of A, B, C, D, E, F, a salt thereof, and a mixture thereof,” it indicates that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

All components and elements positively set forth in this disclosure can be negatively excluded from the claims. In other words, the personal care compositions of the present disclosure can be free or essentially free of all components and elements positively recited throughout the present disclosure. In some instances, the personal care compositions of the present disclosure may be substantially free of non-incidental amounts of the ingredient(s) or compound(s) described herein. A non-incidental amount of an ingredient or compound is the amount of that ingredient or compound that is added into the personal care composition by itself. For example, a personal care composition may be substantially free of a non-incidental amount of an ingredient or compound, although such ingredient(s) or compound(s) may be present as part of a raw material that is included as a blend of two or more compounds.

Some of the various categories of components identified may overlap. In such cases where overlap may exist and the personal care composition includes both components (or the composition includes more than two components that overlap), an overlapping compound does not represent more than one component. For example, certain compounds may be characterized as both an emulsifier and a surfactant. If a particular personal care includes both an emulsifier and a surfactant, the compounds that may be characterized as both an emulsifier and a surfactant will serve only as either the emulsifier or the surfactant—not both.

For readability purposes, the chemical functional groups are in their adjective form; for each of the adjective, the word “group” is assumed. For example, the adjective “alkyl” without a noun thereafter, should be read as “an alkyl group”.

As used herein, the term “antiperspirant” refers to a product applied topically that reduces the production of perspiration (sweat) at that site. As used herein the term “reduce” in the “reduce sweat” context, refers to decrease or lessen of the dampness, perspiration, or wetness in the applied area. As used herein, the term “deodorant” refers to inhibiting the odor causing bacteria that makes the sweat smell.

Aspects of the present invention are directed to personal care composition that may be in the form of a deodorant and/or an antiperspirant. In accordance with an aspect of the invention, provided is a personal care composition comprising a cosmetically acceptable carrier; an antiperspirant component comprising gluconolactone and/or an acid thereof; and; and optionally a thickening agent. In some embodiments, the antiperspirant component further comprises an ingredient selected from: lactobionic acid, maltobionic acid, galactonolactone, glucuronolactone, galacturonolactone, gulonolactone, ribonolactone, saccharic acid lactone, pantolactone, glucoheptonolactone, mannonolactone, galactoheptonolactone, an acid thereof; and a salt thereof, or a combination of two or more thereof. In accordance with another aspect of the invention, provided is personal care composition comprising an antiperspirant component comprising gluconolactone and/or an acid thereof; a thickening agent; from about 0.1 to about 25 wt. % of a polyol, based on the total weight of the personal care composition; and water. In accordance with a further aspect of the invention, provided is a personal care composition comprising an antiperspirant component consisting essentially of gluconolactone and/or an acid thereof.

The personal care compositions may provide a reduction of sweat of about 40% or more. For example, the personal care compositions may provide a reduction of sweat of about 40% or more in comparison to a personal care composition having the same formulation, expect that it includes additional carrier (e.g. water) instead of gluconolactone, lactobionic acid, maltobionic acid, galactonolactone, glucuronolactone, galacturonolactone, gulonolactone, ribonolactone, saccharic acid lactone, pantolactone, glucoheptonolactone, mannonolactone, galactoheptonolactone, an acid thereof, a salt thereof, or a combination of two or more thereof. In certain embodiments, the personal care compositions provide a reduction of sweat of about 45% or more, about 50% or more, about 53% or more, about 59% or more, about 62% or more, about 64% or more, about 66% or more, or about 68% or more. For example, the personal care composition may provide reduction of sweat of about 50 to about 68%, about 53 to about 68%, about 56 to about 68%, about 59 to about 68%, about 62 to about 68%; from about 50 to about 65%, about 53 to about 65%, about 56 to about 65%, about 59 to about 65%, about 62 to about 65%; from about 50 to about 62%, about 53 to about 62%, about 56 to about 62%, about 59 to about 62%, or any range or subrange thereof. The reduction of sweat disclosed above may be achieved over a 24-hour period of application.

The personal care composition may achieve the reduction of sweat while being substantially free or free of certain traditional antiperspirant compositions, such as aluminum based antiperspirant actives, zirconium-based antiperspirant actives, zinc-based antiperspirant actives, titanium-based antiperspirant actives, iron-based antiperspirant actives, and/or magnesium-based antiperspirant actives. For example, the personal care compositions may have about 3 wt. % or less, about 2 wt. % or less, about 1 wt. % or less, about 0.5 wt. % or less, about 0.1 wt. % or less, about 0.05 wt. % or less, or about 0.01 wt. % or less of aluminum-based antiperspirant actives, magnesium-based antiperspirant actives, titanium-based antiperspirant actives, free-from zirconium-based antiperspirant actives, iron-based antiperspirant actives, and/or zinc-based antiperspirant actives (individually or cumulatively), based on the total weight of the personal care composition. In some embodiments, the personal care compositions have about 0 wt. % or 0 wt. % of aluminum-based antiperspirant actives, magnesium-based antiperspirant actives, titanium-based antiperspirant actives, zirconium-based antiperspirant actives, and/or zinc-based antiperspirant actives (individually or cumulatively), based on the total weight of the personal care composition.

As used herein, the term “free of aluminum-based antiperspirant actives”, means that the personal care compositions of the present disclosure do not comprise aluminum-based antiperspirant actives. For example, aluminum-based antiperspirant actives are not added to the personal care compositions of the invention to display some personal care effect.

As used herein, the term “aluminum free” or “free-from aluminum” means that the personal care composition does not contain any aluminum-based antiperspirant. Non limiting examples of aluminum-based antiperspirant actives, can include those listed in the US antiperspirant monograph, such as, aluminum chlorohydrate, aluminum chloride, aluminum dichlorohydrate, aluminum sesquichlorohydrate poylethylene glycol, aluminum sesquichlorohydrate propylene glycol, aluminum zirconium octachlorohydrate, aluminum zirconium pentachlorohydrate, aluminum zirconium tetrachlorohydrate, aluminum zirconium trichlorohydrate, aluminum sesquichlorohydrate, aluminum-zirconium glycine complex (for example, aluminum zirconium trichlorohydrex gly, aluminum zirconium pentachlorohydrex gly, aluminum zirconium tetrachlorohydrex gly and aluminum zirconium octochlorohydrex gly), aluminum chlorohydrex propylene glycol (aluminum chlorohydrex PG), aluminum chlorohydrex polyethylene glycol (aluminum chlorohydrex PEG), aluminum dichlorohydrex propylene glycol (aluminum dichlorohydrex PG), and aluminum dichlorohydrex polyethylene glycol (aluminum dichlorohydrex PEG). In some embodiments, the personal care composition may be free of one or more of the foregoing examples of aluminum-based antiperspirant actives.

As used herein, the term “zirconium free” or “free-from zirconium-based antiperspirant actives” means that the personal care composition does not contain zirconium-based antiperspirant. Non-limiting examples of zirconium-based antiperspirant actives, include aluminum zirconium octachlorohydrate, aluminum zirconium pentachlorohydrate, aluminum zirconium tetrachlorohydrate, aluminum zirconium trichlorohydrate, and aluminum-zirconium glycine complex (e.g., aluminum zirconium trichlorohydrex gly, aluminum zirconium pentachlorohydrex gly, aluminum zirconium tetrachlorohydrex gly and aluminum zirconium octochlorohydrex gly). In some embodiments, the personal care composition may be free of one or more of the foregoing examples of zirconium-based antiperspirant actives.

As used herein, the term “free of zinc-based antiperspirant actives” means that the personal care composition does not comprise zinc-based antiperspirant actives. Non-limiting examples of zinc-based antiperspirant actives can include one or more of zinc oxide, zinc hydroxide, zinc hydroxide ions with counter ions, and zinc ions with counter ions. In some embodiments, the personal care composition may be free of one or more of the foregoing examples of zinc-based antiperspirant actives.

As used herein, the term “free of iron-based antiperspirant actives”, means that the personal care composition does not comprise iron-based antiperspirant actives.

As used herein, the term “free of titanium-based antiperspirant actives” means that the personal care composition does not comprise titanium-based antiperspirant actives. For example, the personal care compositions may include a reduced amount, be substantially free of, or free of titanium-based antiperspirant actives, such as ammonium titanium lactate and the like. In some embodiments, the personal care composition may be free of one or more of the foregoing examples of titanium-based antiperspirant actives.

As used herein, the term “free of magnesium-based antiperspirant actives”, means that the personal care composition does not comprise magnesium-based antiperspirant actives. Non-limiting examples of magnesium-based actives include, but are not limited to, magnesium chloride, magnesium bromide, magnesium fluoride and organic salts such as various alkyl chain length substituted carboxylic acids, magnesium oxide, and magnesium hydroxide. As used herein, the term “axilla” or “axillary region” refers to the armpit or underarm of the body. In some embodiments, the personal care composition may be free of one or more of the foregoing examples of magnesium-based antiperspirant actives.

Additionally or alternatively, the personal care compositions may inhibit and/or reduce the growth of detrimental bacteria, responsible for the underarm malodor. Without being limited to any particular theory, it is believed that by reducing the underarm sweat, the personal care compositions may reduce the formation of the detrimental bacteria responsible for the malodor.

Suitable components, such as those listed below, may be included or excluded from the formulations for the personal care compositions care compositions depending on the specific combination of other components, the form of the personal care compositions, and/or the use of the formulation (e.g., a roll-on, stick, spray, etc.). For example, the personal care composition may be an antiperspirant or deodorant in the form of a roll-on, a gel, a cream, a stick, or an aerosol formulation. The personal care composition may also be in the form of a solid, semi-solid, or liquid composition.

The personal care composition includes an antiperspirant component comprising gluconolactone, lactobionic acid, maltobionic acid, galactonolactone, glucuronolactone, galacturonolactone, gulonolactone, ribonolactone, saccharic acid lactone, pantolactone, glucoheptonolactone, mannonolactone, galactoheptonolactone, an acid thereof, a salt thereof, or a combination of two or more thereof, preferably in an effective amount. The antiperspirant component may comprise gluconolactone, galactonolactone, glucuronolactone, galacturonolactone, gulonolactone, ribonolactone, saccharic acid lactone, pantolactone, glucoheptonolactone, mannonolactone, galactoheptonolactone, an acid thereof, or a combination of two or more thereof. Additionally or alternatively, the antiperspirant component may comprise lactobionic acid, maltobionic acid, gluconic acid, a salt thereof, or a combination of two or more thereof. In some preferred embodiments, the personal care composition includes gluconolactone and/or a salt thereof. The personal care composition may include gluconolactone in an acid form (e.g., gluconic acid). The gluconolactone may be glucanodeltalactone. In some preferred embodiments, the personal care composition includes an antiperspirant component consisting of one or more compound(s) selected from gluconolactone, lactobionic acid, maltobionic acid, galactonolactone, glucuronolactone, galacturonolactone, gulonolactone, ribonolactone, saccharic acid lactone, pantolactone, glucoheptonolactone, mannonolactone, galactoheptonolactone, an acid thereof, a salt thereof.

The total amount of gluconolactone, lactobionic acid, maltobionic acid, galactonolactone, glucuronolactone, galacturonolactone, gulonolactone, ribonolactone, saccharic acid lactone, pantolactone, glucoheptonolactone, mannonolactone, galactoheptonolactone, an acid thereof, a salt thereof, or a combination of two or more thereof present in the personal care composition may be from about 0.2 to about 11 wt. %, based on the total weight of the personal care composition. For example, the personal care composition may include gluconolactone, lactobionic acid, maltobionic acid, galactonolactone, glucuronolactone, galacturonolactone, gulonolactone, ribonolactone, saccharic acid lactone, pantolactone, glucoheptonolactone, mannonolactone, galactoheptonolactone, an acid thereof, a salt thereof, or a combination of two or more thereof in a total amount from about 0.2 to about 9 wt. %, about 0.2 to about 7 wt. %, about 0.2 to about 5 wt. %, about 0.2 to about 4 wt. %, about 0.2 to about 3 wt. %, about 0.2 to about 2 wt. %, about 0.2 to about 1 wt. %; from about 0.5 to about 11 wt. %, about 0.5 to about 9 wt. %, about 0.5 to about 7 wt. %, about 0.5 to about 5 wt. %, about 0.5 to about 4 wt. %, about 0.5 to about 3 wt. %, about 0.5 to about 2 wt. %, about 0.5 to about 1 wt. %; from about 1 to about 11 wt. %, about 1 to about 9 wt. %, about 1 to about 7 wt. %, about 1 to about 5 wt. %, about 1 to about 4 wt. %, about 1 to about 3 wt. %, about 1 to about 2 wt. %; from about 2 to about 11 wt. %, about 2 to about 9 wt. %, about 2 to about 7 wt. %, about 2 to about 5 wt. %, about 2 to about 4 wt. %, about 2 to about 3 wt. %; from about 3 to about 11 wt. %, about 3 to about 9 wt. %, about 3 to about 7 wt. %, about 3 to about 5 wt. %; from about 5 to about 11 wt. %, about 5 to about 9 wt. %, about 5 to about 7 wt. %; from about 7 to about 11 wt. %, about 7 to about 9 wt. %, about 9 to about 11 wt. %, or any range or subrange thereof, based on the total weight of the personal care composition.

The personal care composition may include an additional polyhydroxy acid and/or salt thereof other than gluconic acid. The amount of additional polyhydroxy acid and/or salt thereof may be present in an amount from about 0.1 to about 10 wt. %, based on the total weight of the personal care composition. For example, the amount of additional polyhydroxy acid and/or salt thereof present in the personal care composition may be from about 0.1 to about 8 wt. %, about 0.1 to about 6 wt. %, about 0.1 to about 5 wt. %, about 0.1 to about 4 wt. %, about 0.1 to about 3 wt. %, about 0.1 to about 2 wt. %, about 0.1 to about 1 wt. %; from about 0.5 to about 8 wt. %, about 0.5 to about 6 wt. %, about 0.5 to about 5 wt. %, about 0.5 to about 4 wt. %, about 0.5 to about 3 wt. %, about 0.5 to about 2 wt. %, about 0.5 to about 1 wt. %; about 1 to about 10 wt. %, about 1 to about 8 wt. %, about 1 to about 6 wt. %, about 1 to about 5 wt. %, about 1 to about 4 wt. %, about 1 to about 3 wt. %, about 1 to about 2 wt. %; about 2 to about 10 wt. %, about 2 to about 8 wt. %, about 2 to about 6 wt. %, about 2 to about 5 wt. %, about 2 to about 4 wt. %, about 2 to about 3 wt. %; about 3 to about 10 wt. %, about 3 to about 8 wt. %, about 3 to about 6 wt. %, about 3 to about 5 wt. %, about 3 to about 4 wt. %, including ranges and subranges thereof, based on the total weight of the personal care composition. The polyhydroxy acids may include, but are not limited to, gluconolactone, galactose, lactobionic acid, or combinations thereof. In some embodiments, the polyhydroxy acids are lactobionic acid or combinations thereof.

The personal care composition can include one or more alpha hydroxy acid(s) and/or a salt thereof. For example, the personal care composition may include one or more one or more alpha hydroxy acid(s), such as those selected from C₃ to C₇ alpha-hydroxy acid or C₄ to C₆ alpha-hydroxy acid. In some embodiments, the personal care composition includes a salt of an alpha-hydroxy acid. The salt of the alpha-hydroxy acid is preferably a sodium salt or a potassium salt. In at least one embodiment, the salt is a sodium salt (i.e., the cation associated with the salt is a sodium). Non-limiting examples of alpha hydroxy acids include but are not limited to, mandelic acid, glycolic acid, citric acid, lactic acid, malic acid, tartaric acid, phytic acid, hydroxycaprylic acid, hydroxycapric acid, glutaric acid, gluconic acid, a salt thereof, or a combination of two or more thereof. In some embodiments, the alpha hydroxy acids are citric acid, mandelic acid, glycolic acid, lactic acid, a salt thereof, or a combination of two or more thereof. In certain instance, the alpha-hydroxy acid is selected from the group consisting of: malic acid, tartaric acid, alpha-hydroxy glutaric acid, gluconic acid, a salt thereof, and a combination of two or more thereof. Yet in further instances, the alpha-hydroxy acid and/or salt thereof is selected from the group consisting of: lactic acid, malic acid, and a combination thereof. In at least one embodiment, the alpha hydroxy acid is lactic acid.

The personal care compositions of the invention may comprise alpha hydroxy acid(s) and/or salt(s) thereof in a total amount from about 0.1 to about 7 wt. %, based on the total weight of the personal care composition. In some instances, the one or alpha hydroxy acid(s) and/or salt(s) thereof is present in the personal care composition in an amount from about 0.1 to about 6 wt. %, about 0.1 to about 5 wt. %, about 0.1 to about 4 wt. %, about 0.1 to about 3 wt. %, about 0.1 to about 2 wt. %, about 0.1 to about 1 wt. %; from about 0.4 to about 7 wt. %, about 0.4 to about 6 wt. %, about 0.4 to about 5 wt. %, about 0.4 to about 4 wt. %, about 0.4 to about 3 wt. %, about 0.4 to about 2 wt. %, about 0.4 to about 1 wt. %; from about 0.8 to about 7 wt. %, about 0.8 to about 6 wt. %, about 0.8 to about 5 wt. %, about 0.8 to about 4 wt. %, about 0.8 to about 3 wt. %, about 0.8 to about 2 wt. %; from about 1.4 to about 7 wt. %, about 1.4 to about 6 wt. %, about 1.4 to about 5 wt. %, about 1.4 to about 4 wt. %, about 1.4 to about 3 wt. %; from about 2 to about 7 wt. %, about 2 to about 6 wt. %, about 2 to about 5 wt. %, about 2 to about 4 wt. %, about 2 to about 3 wt. %; from about 3 to about 7 wt. %, about 3 to about 6 wt. %, about 3 to about 5 wt. %; from about 4 to about 7 wt. %, about 4 to about 6 wt. %, about 5 to about 7 wt. %, or any range or subrange thereof, based on the total weight of the personal care composition.

The personal care compositions may include one or more emollient(s). The amount of emollient(s) in the personal care composition may be from about 0.1 to about 15 wt. %, based on the total weight of the personal care composition. For example, the total amount of emollient(s) in the personal care composition may be about 0.1 to about 15 wt. %, about 0.1 to about 12 wt. %, about 0.1 to about 9 wt. %, about 0.1 to about 6 wt. %, about 0.1 to about 4 wt. %, about 0.1 to about 2 wt. %; from about 0.5 to about 15 wt. %, about 0.5 to about 12 wt. %, about 0.5 to about 9 wt. %, about 0.5 to about 6 wt. %, about 0.5 to about 4 wt. %, about 0.5 to about 2 wt. %; from about 1 to about 15 wt. %, about 1 to about 12 wt. %, about 1 to about 9 wt. %, about 1 to about 6 wt. %, about 1 to about 4 wt. %, about 1 to about 2 wt. %; from about 2 to about 15 wt. %, about 2 to about 12 wt. %, about 2 to about 9 wt. %, about 2 to about 6 wt. %, about 2 to about 4 wt. %; from about 4 to about 15 wt. %, about 4 to about 12 wt. %, about 4 to about 9 wt. %, about 4 to about 6 wt. %; from about 7 to about 15 wt. %, about 7 to about 12 wt. %, about 7 to about 9 wt. %; from about 10 to about 15 wt. %, about 10 to about 12 wt. %, or about 12 to about 15 wt. %, including any range or subrange thereof, based on the total weight of the personal care composition.

Classes of non-volatile emollients include non-silicone and silicone emollients. Non-volatile, non-silicone emollients include C₁₂₋₁₅ alkyl benzoate. The one or more emollient(s) may be selected from the group consisting of: isododecane, sodium starch octenylsuccinate, C₁₂₋₁₅ alkyl benzoate, mineral oil, caprylyl glycol, dimethicone, dicaprylyl ether, ethyl trisiloxane, PPG-14 butyl ether, PPG-3 myristyl ether, stearyl alcohol, stearic acid and salts thereof, glyceryl monoricinoleate, isobutyl palmitate, isocetyl stearate, isocetyl stearate, sulphated tallow, oleyl alcohol, propylene glycol, isopropyl laurate, mink oil, sorbitan stearate, cetyl alcohol, hydrogenated castor oil, stearyl stearate, hydrogenated soy glycerides, isopropyl isostearate, hexyl laurate, dimethyl brassylate, decyl oleate, diisopropyl adipate, n-dibutyl sebacate, diisopropyl sebacate, 2-ethyl hexyl palmitate, isononyl isononanoate, isodecyl isononanoate, isotridecyl isononanoate, 2-ethyl hexyl palmitate, 2-ethyl hexyl stearate, Di-(2-ethyl hexyl) adipate), Di-(2-ethyl hexyl) succinate, isopropyl myristate, isopropyl palmitate, isopropyl stearate, octacosanol, butyl stearate, polyethylene glycols, oleic acid, triethylene glycol, lanolin, castor oil, acetylated lanolin alcohols, acetylated lanolin, petrolatum, isopropyl ester of lanolin, fatty acids, mineral oils, butyl myristate, isostearic acid, palmitic acid, PEG-8 distearate, PEG-23 oleyl ether, olelyl oleate, isopropyl linoleate, cetyl lactate, lauryl lactate, myristyl lactate, quaternised hydroxy alkyl, aminogluconate, vegetable oils, isodecyl oleate, isostearyl neopentanoate, myristyl myristate, oleyl ethoxy myristate, diglycol stearate, ethylene glycol monostearate, myristyl stearate, isopropyl lanolate, paraffin waxes, glycyrrhizic acid, hydrocyethyl stearate amide, and a combination of two or more thereof.

The personal care composition may include one or more polyol(s). Preferably, the personal care compositions include one or more polyol(s) in an amount from about 0.1 to about 25 wt. %, based on the total weight of the personal care composition. For example, the total amount of polyol(s) in the personal care composition may be from about 0.1 to about 22 wt. %, about 0.1 to about 18 wt. %, about 0.1 to about 12 wt. %, about 0.1 to about 8 wt. %, about 0.1 to about 6 wt. %, about 0.1 to about 4 wt. %, about 0.1 to about 3 wt. %, about 0.1 to about 2 wt. %; about 0.1 to about 1 wt. %; from about 0.5 to about 22 wt. %, about 0.5 to about 18 wt. %, about 0.5 to about 12 wt. %, about 0.5 to about 8 wt. %, about 0.5 to about 6 wt. %, about 0.5 to about 4 wt. %, about 0.5 to about 3 wt. %, about 0.5 to about 2 wt. %; about 0.5 to about 1 wt. %; from about 1 to about 22 wt. %, about 1 to about 18 wt. %, about 1 to about 12 wt. %, about 1 to about 8 wt. %, about 1 to about 6 wt. %, about 1 to about 4 wt. %, about 1 to about 3 wt. %, about 1 to about 2 wt. %; from about 2 to about 22 wt. %, about 2 to about 18 wt. %, about 2 to about 12 wt. %, about 2 to about 8 wt. %, about 2 to about 6 wt. %, about 2 to about 4 wt. %, about 2 to about 3 wt. %; from about 3 to about 22 wt. %, about 3 to about 18 wt. %, about 3 to about 12 wt. %, about 3 to about 8 wt. %, about 3 to about 6 wt. %; from about 5 to about 25 wt. %, about 5 to about 22 wt. %, about 5 to about 20 wt. %, about 5 to about 18 wt. %, about 5 to about 16 wt. %, about 5 to about 12 wt. %, about 5 to about 10 wt. %, about 5 to about 8 wt. %; from about 7 to about 25 wt. %, about 7 to about 22 wt. %, about 7 to about 20 wt. %, about 7 to about 18 wt. %, about 7 to about 16 wt. %; from about 9 to about 25 wt. %, about 9 to about 22 wt. %, about 9 to about 20 wt. %, about 9 to about 18 wt. %, about 9 to about 16 wt. %; from about 12 to about 25 wt. %, about 12 to about 22 wt. %, about 12 to about 20 wt. %, about 12 to about 18 wt. %, about 12 to about 16 wt. %; from about 14 to about 25 wt. %, about 14 to about 22 wt. %, about 14 to about 20 wt. %, about 14 to about 18 wt. %; from about 16 to about 25 wt. %, about 16 to about 22 wt. %, about 16 to about 20 wt. %; from about 18 to about 25 wt. %, about 21 to about 25 wt. %, or any range or subrange thereof, based on the total weight of the personal care composition.

The polyol(s) may be chosen from glycols or compounds with numerous hydroxyl groups. In some cases, the one or more polyols is/are selected from the group consisting of C₂-C₃₂ polyols. The one or more polyols may be liquid at ambient temperature (25° C.). The one or more polyols may have from 2 to 32 carbon atoms, from 3 to 16 carbon atoms, or from 3 to 12 carbon atoms.

Polyols that may be included in the personal care composition, in certain instances, include ethylene glycol, propylene glycol, butylene glycol, hexylene glycol, glycerin, diglycerin, diethylene glycol, dipropylene glycol, and mixtures thereof. In some cases, the personal care composition comprises glycerin, and optionally one or more polyols other than glycerin. Non-limiting examples of polyols that may, optionally, be included in the personal care include and/or may be chosen from alkanediols such as glycerin, 1,2,6-hexanetriol, trimethylolpropane, ethylene glycol, propylene glycol, butylene glycol, pentylene glycol, hexylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, dipropylene glycol mono-iso-propyl ether, sorbitol, sorbitan, triacetin, and a mixture thereof.

The one or more polyols may, optionally, be glycols or glycol ethers such as, e.g., monomethyl, monoethyl and monobutyl ethers of ethylene glycol, propylene glycol or ethers thereof such as, e.g., monomethyl ether of propylene glycol, butylene glycol, hexylene glycol, dipropylene glycol as well as alkyl ethers of diethylene glycol, e.g., monoethyl ether or monobutyl ether of diethylene glycol. In some cases, the one or more polyols may include or be chosen from ethylene glycol, propylene glycol, butylene glycol, hexylene glycol, pentylene glycol, 1,3-propanediol, diethylene glycol, dipropylene glycol, 1,4-butanediol, 1,5-pentanediol, hexane-1,6-diol, glycerin, diglycerin, caprylyl glycol, and/or a mixture of two or more thereof. In certain embodiments, the personal care composition may include polyol(s) selected from the group consisting of: glycerin (preferably, vegetable refined glycerin), butylene glycol, and 1,3-propanediol, polyglutamic acid, saccharide isomerate, and combinations thereof. In some embodiments, the polyol is a polyethylene glycol, such as polyethylene glycol 600.

Additionally or alternatively, the personal care composition may include polyol(s) having a molecular weight of from about 100 to 5000 g/mol. For instance, the polyol may comprise a polyethylene glycol, a polypropylene glycol, a block polymer of polyethylene glycol and polypropylene glycol, or a combination of two or more thereof. In some embodiments, the polyol comprises a polypropylene glycol, a polypropylene glycol, and/or a block polymer of polyethylene glycol and polypropylene glycol having a molecular weight of about 100 to about 900, about 200 to about 800, about 400, about 1500 to about 2500, about 2000 to about 4500 or any range or subrange thereof.

The personal care composition may optionally include one or more thickening agent(s). The total amount of thickening agent(s) in the personal care composition, if present, may be from about 0.1 to about 7 wt. %, about 0.1 to about 5 wt. %, about 0.1 to about 4 wt. %, about 0.1 to about 3 wt. %, about 0.1 to about 2 wt. %, about 0.1 to about 1 wt. %; from about 0.5 to about 7 wt. %, about 0.5 to about 5 wt. %, about 0.5 to about 4 wt. %, about 0.5 to about 3 wt. %, about 0.5 to about 2 wt. %, about 0.5 to about 1 wt. %; from about 1 to about 7 wt. %, about 1 to about 5 wt. %, about 1 to about 4 wt. %, about 1 to about 3 wt. %, about 1 to about 2 wt. %; from about 2 to about 7 wt. %, about 2 to about 5 wt. %, about 2 to about 4 wt. %, about 2 to about 3 wt. %; from about 3 to about 7 wt. %, about 3 to about 5 wt. %, about 3 to about 4 wt. %, or any range or subrange thereof, based on the total weight of the personal care composition.

Thickening agents may be referred to as “thickeners.” Thickening agents are typically included to increase the viscosity of the personal care compositions. Non-limiting examples of thickening agents that may be incorporated into or excluded from the personal care compositions include natural gums, polyacrylate crosspolymers or cross linked polyacrylate polymers, cationic acrylate copolymers, anionic acrylic or carboxylic acid polymers, polyacrylamide polymers, polysaccharides such as cellulose derivatives, cationic gums, polyquaterniums, vinylpyrrolidone homopolymers/copolymers, C₈₋₂₄ hydroxyl substituted aliphatic acid, C₈₋₂₄ conjugated aliphatic acid, sugar fatty esters, polyglyceryl esters, and a mixture thereof. Particular types of thickening agents that may be mentioned include: gums (e.g., natural gums), sucrose esters, polyvinylpyrrolidone (PVP) and co-polymers, celluloses, polyquaternium compounds, and carboxylic acid or carboxylate based homopolymer or co-polymer, which can be linear or crosslinked.

The thickening agents may, also or alternatively, be chosen from xanthan gum, guar gum, biosaccharide gum, cellulose, acacia seneca gum, sclerotium gum, agarose, pectin, gellan gum, hyaluronic acid. In some instances, the thickening agent may be a gum, such as one or more selected from the group consisting of: xanthan gum, sodium starch octenylsuccinate, hydroxypropyl starch phosphate, diutan gum, carrageenan, carboxymethylcellulose, cellulose gum, sclerotium gum, hydroxypropyl methylcellulose, and combinations thereof. Additionally, the thickening agents may include polymeric thickeners selected from the group consisting of: ammonium polyacryloyldimethyl taurate, ammonium acryloyldimethyltaurate NP copolymer, acrylates copolymers, polyacrylamide, carbomer, acrylates/C₁₀₋₃₀ alkyl acrylate crosspolymer, acrylates/beheneth-25 methacrylate copolymer, and a mixture thereof.

In certain cases, the personal care composition may comprise a thickening agent selected from the group consisting of: thickening polymers, such as polyvinyl alcohol, sodium polyacrylate, sodium polymethacrylate, polyacrylic acid glycerin ester, carboxyvinyl polymer, polyacrylamide, polyvinyl pyrrolidone, polyvinyl methylether, polyvinyl sulfone, maleic acid copolymer, polyethylene oxide, polydiallyl amine, polyethylene imine, water soluble cellulose derivatives (for example, carboxymethyl cellulose, methyl cellulose, methylhydroxypropyl cellulose, hydroxyethyl cellulose, hydroxypropyl cellulose, cellulose sulfate sodium salt), and/or starch derivatives (for example, starch oxide, dialdehyde starch, dextrin, British gum, acetyl starch, starch phosphate, carboxymethyl starch, hydroxyethyl starch, hydroxypropyl starch). In some embodiments, the thickening agent is selected from the group consisting of: among hydroxypropyl methylcellulose, polyethylene glycols (PEGs), polyacrylic acids (sold under the name of Carbopol), cross-linked homopolymer of acrylic acid (sold under the name of Carbopol® Ultrez 30 polymer), and acrylates of C₁₀₋₃₀ alkyl acrylate crosspolymer (sold under the name of Carbopol® Ultrez 20 polymer).

Additionally or alternatively, the personal care compositions may include one or more structuring agent. The one or more structuring agent(s), if present, may be in an amount from about 0.5 to about 9 wt. %, based on the total weight of the personal care composition. For instance, the personal care composition may include one or more structuring agent(s) in an amount from about 0.5 to about 9 wt. %, about 0.5 to about 7 wt. %, about 0.5 to about 6 wt. %, about 0.5 to about 5 wt. %, about 0.5 to about 4 wt. %, about 0.5 to about 3 wt. %, about 0.5 to about 2 wt. %, about 0.5 to about 1.5 wt. %, about 0.5 to about 1 wt. %; from about 1 to about 9 wt. %, about 1 to about 7 wt. %, about 1 to about 6 wt. %, about 1 to about 5 wt. %, about 1 to about 4 wt. %, about 1 to about 3 wt. %, about 1 to about 2 wt. %; from about 1.5 to about 9 wt. %, about 1.5 to about 7 wt. %, about 1.5 to about 6 wt. %, about 1.5 to about 5 wt. %, about 1.5 to about 4 wt. %, about 1.5 to about 3 wt. %, about 1.5 to about 2 wt. %; from about 2 to about 9 wt. %, about 2 to about 7 wt. %, about 2 to about 6 wt. %, about 2 to about 5 wt. %, about 2 to about 4 wt. %, about 2 to about 3 wt. %; from about 3 to about 9 wt. %, about 3 to about 7 wt. %, about 3 to about 6 wt. %, about 3 to about 5 wt. %, about 3 to about 4 wt. %; from about 4 to about 9 wt. %, about 4 to about 7 wt. %, about 4 to about 6 wt. %, about 4 to about 5 wt. %, or any range or subrange thereof, based on the total weight of the personal care composition.

Examples of structuring agents include waxes and/or gelling agents. The structuring agents may be a solid under ambient conditions, such as wax (waxy materials), organic solids, modified silicone solids, crystalline or other gellants, or combinations thereof. In certain embodiments, the structuring agent may be a gelling agent. Examples of structuring agents include waxes, esters of fatty acid and fatty alcohol, triglycerides, or other cosmetically acceptable materials (e.g., which are solid or semi-solid at room temperature and provide a consistency suitable for application to the skin) and combinations thereof. In some embodiments, the structuring agent(s) may be selected from the group consisting of: steareth-21, steareth-10, steareth-11, steareth-13, steareth-15, steareth-20, ceteareth-6, ceteareth-7, ceteareth-8, ceteareth-9, ceteareth-10, ceteareth-11, and ceteareth-12, polyglyceryl-6 palmitate/succinate mixture, glyceryl stearate, cetearyl alcohol (e.g., a 70/30 Stearyl/Cetyl Alcohol Blend), and a combination of two or more thereof.

In certain embodiments, the personal care composition may include one or more preservative(s). The preservative(s) may be selected from the group consisting of: caprylyl glycol, phenoxyethanol, butylated hydroxytoluene, ethylenediaminetetraacetic acid, ethylhexylglycerin, citric acid, benzoic acid, a salt thereof, and combinations thereof. Additional examples of preservatives include lactic acid, sodium benzoate, sodium lactate, triethyl citrate, butylated hydroxytoluene, and pentaerythrityl tetra-di-t-butyl hydroxyhydrocinnamate, a salt thereof, or combinations thereof.

The personal care composition may comprise the preservative(s) in an amount from about 0.1 to about 7 wt. %, based on the total weight of the personal care composition. In some instances, the one or more preservative(s) is present in the personal care composition in a total amount from about 0.1 to about 6 wt. %, about 0.1 to about 5 wt. %, about 0.1 to about 4 wt. %, about 0.1 to about 3 wt. %, about 0.1 to about 2 wt. %, about 0.1 to about 1 wt. %; from about 0.4 to about 7 wt. %, about 0.4 to about 6 wt. %, about 0.4 to about 5 wt. %, about 0.4 to about 4 wt. %, about 0.4 to about 3 wt. %, about 0.4 to about 2 wt. %, about 0.4 to about 1 wt. %; from about 0.8 to about 7 wt. %, about 0.8 to about 6 wt. %, about 0.8 to about 5 wt. %, about 0.8 to about 4 wt. %, about 0.8 to about 3 wt. %, about 0.8 to about 2 wt. %; from about 1.4 to about 7 wt. %, about 1.4 to about 6 wt. %, about 1.4 to about 5 wt. %, about 1.4 to about 4 wt. %, about 1.4 to about 3 wt. %; from about 2 to about 7 wt. %, about 2 to about 6 wt. %, about 2 to about 5 wt. %, about 2 to about 4 wt. %, about 2 to about 3 wt. %; from about 3 to about 7 wt. %, about 3 to about 6 wt. %, about 3 to about 5 wt. %; from about 4 to about 7 wt. %, about 4 to about 6 wt. %, about 5 to about 7 wt. %, or any range or subrange thereof, based on the total weight of the personal care composition.

The personal care composition may comprise water. Water may be present in the personal care composition in an amount ranging from about 45 to about 94 wt. %, based on the total weight of the personal care composition. For example, the personal care composition may comprise water in an amount from about 55 to about 94 wt. %, about 65 to about 94 wt. %, about 68 to about 94 wt. %, about 71 to about 94 wt. %, about 75 to about 94 wt. %, about 78 to about 94 wt. %, about 81 to about 94 wt. %, about 84 to about 94 wt. %; from about 45 to about 91 wt. %, about 55 to about 91 wt. %, about 65 to about 91 wt. %, about 68 to about 91 wt. %, about 71 to about 91 wt. %, about 75 to about 91 wt. %, about 78 to about 91 wt. %, about 81 to about 91 wt. %; from about 45 to about 88 wt. %, about 55 to about 88 wt. %, about 65 to about 88 wt. %, about 68 to about 88 wt. %, about 71 to about 88 wt. %, about 75 to about 88 wt. %, about 78 to about 88 wt. %; from about 45 to about 85 wt. %, about 55 to about 85 wt. %, about 65 to about 85 wt. %, about 68 to about 85 wt. %, about 71 to about 85 wt. %, about 75 to about 85 wt. %, about 78 to about 85 wt. %; from about 45 to about 82 wt. %, about 55 to about 82 wt. %, about 65 to about 82 wt. %, about 68 to about 82 wt. %, about 71 to about 82 wt. %, about 75 to about 82 wt. %; from about 45 to about 79 wt. %, about 55 to about 79 wt. %, about 65 to about 79 wt. %, about 68 to about 79 wt. %; from about 45 to about 76 wt. %, about 55 to about 76 wt. %, about 65 to about 76 wt. %, about 68 to about 76 wt. %, or any range or subrange thereof, based on the total weight of the personal care composition.

The personal care composition may include one or more pH adjusters to increase or decrease the overall pH of the personal care composition. For example, one or more acids may be included to decrease the pH of the personal care composition. Examples of suitable acids for decreasing the pH of the personal care composition include, but are not limited to, citric acid, acetic acid, and the like. The personal care composition may include one or more bases, such as sodium hydroxide, potassium hydroxide and the like, to increase the pH of the personal care composition. Additional or alternative acids and bases that are suitable for adjusting the pH of the personal care composition are readily known to one of ordinary skill in the art.

The amount of the pH adjuster in the personal care composition may be based on the desired pH of the final form of personal care composition and/or product. For example, the total amount of the pH adjuster may range from about 0.05 to about 20 wt. %, based on the total weight of the personal care composition. In some instances, the total amount of pH adjuster is from about 0.05 to about 15 wt. %, about 0.1 to about 10 wt. %, or about 0.12 to about 5 wt. %, including ranges and sub-ranges therebetween, based on the total weight of the personal care composition.

The personal care compositions may have a pH from 4.5 to about 10, 4.5 to about 9, 4.5 to about 8, 4.5 to about 7, 4.5 to about 6; from about 5 to about 10, about 5 to about 9, about 5 to about 8, about 5 to about 7, about 5 to about 6; from about 6 to about 10, about 6 to about 9, about 6 to about 8, or about 6 to about 7; from about 7 to about 10, about 7 to about 9, or about 7 to about 8, including any ranges and subranges therebetween. In some embodiments, the personal care composition has a pH of from about 3.5 to about 7.5. In other embodiments, the personal care composition has a pH of from about 4 to about 7. In further embodiments, the personal care composition has a pH of from about 4.5 to about 6.5. Still other embodiments provide personal care compositions having a pH of from about 5 to about 6.5.

Additional ingredients may be present in the personal care composition. These include water and ingredients to thicken, preserve, emulsify, add fragrance, add pigment and/or color. The personal care compositions may include any of the following additional ingredients in an amount of from about 0.01 to about 5 wt. %. In some instances, the amount of additional ingredients present in the personal care composition is from about 0.01 to about 4 wt. %, about 0.01 to about 3 wt. %, about 0.01 to about 2 wt. %, about 0.01 to about 1 wt. %, about 0.01 to about 0.5 wt. %, about 0.01 to about 0.1 wt. %; about 0.1 to about 5 wt. %, about 0.1 to about 4 wt. %, about 0.1 to about 3 wt. %, about 0.1 to about 2 wt. %, about 0.1 to about 1 wt. %, about 0.1 to about 0.5 wt. %, about 0.1 to about 0.1 wt. %; about 0.5 to about 5 wt. %, about 0.5 to about 4 wt. %, about 0.5 to about 3 wt. %, about 0.5 to about 2 wt. %, about 0.5 to about 1 wt. %; about 0.75 to about 5 wt. %, about 0.75 to about 4 wt. %, about 0.75 to about 3 wt. %, about 0.75 to about 2 wt. %, about 0.75 to about 1 wt. %; about 1 to about 5 wt. %, about 1 to about 4 wt. %, about 1 to about 3 wt. %, about 1 to about 2 wt. %; about 2 to about 5 wt. %, about 2 to about 4 wt. %, about 2 to about 3 wt. %; about 3 to about 5 wt. %, about 3 to about 4 wt. %, including any range or subrange therebetween, based on the total weight of the personal care composition.

Non-limiting examples of fragrances and perfumes include odor compounds selected from the group consisting of: 7-acetyl-1,2,3,4,5,6,7,8-octahydro-1,1,6,7-tetramethylnaphthalene, α-ionone, β-ionone, γ-ionone α-isomethylionone, methylcedrylone, methyl dihydrojasmonate, methyl 1,6,10-trimethyl-2,5,9-cyclododecatrien-1-yl ketone, 7-acetyl-1,1,3,4,4,6-hexamethyltetralin, 4-acetyl-6-tert-butyl-1,1-dimethylindane, hydroxyphenylbutanone, benzophenone, methyl β-naphthyl ketone, 6-acetyl-1,1,2,3,3,5-hexamethylindane, 5-acetyl-3-isopropyl-1,1,2,6-tetramethylindane, 1-dodecanal, 4-(4-hydroxy-4-methylpentyl)-3-cyclohexene-1-carboxaldehyde, 7-hydroxy-3,7-dimethyloctanal, 10-undecen-1-al, isohexenylcyclohexylcarboxaldehyde, formyltricyclodecane, condensation products of hydroxycitronellal and methyl anthranilate, condensation products of hydroxycitronellal and indole, condensation products of phenylacetaldehyde and indole, 2-methyl-3-(para-tert-butylphenyl) propionaldehyde, ethylvanillin, heliotropin, hexylcinnamaldehyde, amylcinnamaldehyde, 2-methyl-2-(isopropylphenyl) propionaldehyde, coumarin, γ-decalactone, cyclopentadecanolide, 1,3,4,6,7,8-hexahydro-4,6,6,7,8,8-hexamethylcyclopenta-γ-2-benzopyran, β-naphthol methyl ether, ambroxane, dodecahydro-3a,6,6,9a-tetramethylnaphtho [2,1b]furan, cedrol, 5-(2,2,3-trimethylcyclopent-3-enyl)-3-methylpentan-2-ol, 2-ethyl-4-(2,2,3-trimethyl-3-cyclopenten-1-yl)-2-buten-1-ol, caryophyllene alcohol, tricyclodecenyl propionate, tricyclodecenyl acetate, benzyl salicylate, cedryl acetate, and tert-butylcyclohexyl acetate.

Other fragrances may include odor compounds selected from the group consisting of: essential oils, resinoids and resins from a large number of sources, such as, for example, Peru balsam, olibanum resinoid, styrax, labdanum resin, nutmeg, cassia oil, benzoin resin, coriander, and lavandin.

The personal care composition may further comprise one or more colorants. The colorants may be a pigment, a dye, or mixtures thereof. Non-limiting examples of pigments include titanium dioxide, Zinc Oxide, Kaolin, Mica etc. Non-limiting examples of dyes include food dyes suitable for food, drug and cosmetic applications, and mixtures thereof. Some color agents (colorants) are known as FD&C dyes.

The colorants may be present in an amount ranging from about 0.0001 to about 0.4 wt. %, including all percentages and subranges therebetween, based on the total weight of the personal care composition. In some embodiments, the colorants may be present in an amount ranging from about 0.0001 to about 4 wt. %, including all percentages and subranges therebetween, based on the total weight of the personal care composition.

Further embodiments of the present invention provide personal care compositions wherein the antiperspirant component comprises gluconolactone, or an acid thereof; and a metal oxalate. In some embodiments, the metal oxalate is selected from potassium oxalate; potassium titanium oxide oxalate; and a combination thereof. In some embodiments, the metal in the metal oxalate is selected from the group consisting of: Ba, Ti, V, Cr, Mn, Fe, Co, Ni, Cu, Re, Os, Ir, and a combination of two or more thereof.

As used herein, “potassium titanium oxalate” refers to compositions/compounds with a molecular formula of: C₄K₂O₉Ti (or C₄K₂O₉Ti·2H₂O if in dihydrate form). As used here, “potassium titanium oxalate” may be abbreviated using the designation “KTO”.

In some embodiments, the present invention provides a method for treating hyperhidrosis in a mammalian subject, comprising applying a personal care composition as described herein to a skin surface of a mammalian subject in need thereof. In other embodiments, the present invention provides a method for reducing or preventing sweat production in a mammalian subject, comprising applying a personal care composition as described herein, to a skin surface of a mammalian subject in need thereof.

Some embodiments of the present invention provide a method for controlling perspiration and/or odor. In some embodiments, the method typically includes applying to a user's skin an antiperspirant effective amount of a personal care composition disclosed herein.

### EXAMPLES

### Example 1

An exemplary personal care composition (Composition A) of the present invention may have a formulation shown in Table 1.

TABLE 1

| Material | Comp. A (Wt. %) |
|---|---|
| Gluconolactone and/or an acid thereof | 2 |
| Glycerin | 15 |
| Isododecane | 1 |
| Emollients | 0.7 |
| Preservative | 1.3 |
| Inulin | 0.2 |
| Antibacterial | 0.9 |
| Polyhydric alcohol | 0.5 |
| Gum Base Blend* | 2 |
| Calcium Silicate | — |
| Triethyl Citrate | 0.5 |
| Fragrance | 1.4 |
| Water | Q.S. to 100 |

### Example 2

A personal care composition (Composition B) having a gum base backbone was prepared in accordance with aspects of the invention. Composition B included 2 wt. % of gluconolactone. A comparative composition (Comparative Composition 1) was prepared having the same formulation as Composition B, except that the Comparative Composition 1 had an additional 2 wt. % of water instead of gluconolactone. The formulation of Composition B and Comparative Composition 1 are shown in Table 2 (below).

TABLE 2

| | Comparative | |
|---|---|---|
| US INCI Name | Comp. 1 (wt. %) | Comp. B (wt. %) |
| WATER | Q.S. to 100 | Q.S. to 100 |
| Glycerin | 15 | 15 |
| Gluconolactone | — | 2 |
| Sodium Lactate | 1.5 | 1.5 |
| Fragrance | 1.4 | 1.4 |
| Thickener(s) (e.g., | 2 | 2 |
| Xanthan Gum) | | |
| Preservative | 1.4 | 1.4 |
| Emollients | 1.5 | 1.5 |
| Triethyl Citrate | 0.5 | 0.5 |
| Polyhydric Alcohol | 0.5 | 0.5 |
| Inulin | 0.25 | 0.25 |
| Emollient | 0.2 | 0.2 |

Composition B and Comparative Composition 1 were evaluated to assess sweat reduction. Specifically, a thermal imaging study was conducted to evaluate the performance and efficacy of Composition B. The thermal imaging study simulates the performance of Composition B and Comparative Composition 1 under real-life conditions. The baseline was collected by having a human subject perform aerobic exercise in a 100% cotton T-shirt. Immediately afterwards, the T-shirt was collected and the sweat retained was recorded using a thermal camera. On the following day, Composition B was applied to the one of the subject's underarms at least 2 hours prior to exercise while Comparative Composition 1 was applied to the other underarm of the subject. Thermal imaging was then conducted.

The thermal imaging results indicated that Composition B provided a reduction of sweat of about 62% relative to Comparative Composition 1.

What is claimed is:

1. A personal care composition comprising:
a cosmetically acceptable carrier;
an antiperspirant component comprising gluconolactone; and
optionally, a thickening agent.

2. The personal care composition according to claim 1, wherein the antiperspirant component further comprises an ingredient selected from: maltobionic acid, lactobionic acid, galactonolactone, glucuronolactone, galacturonolactone, gulonolactone, ribonolactone, saccharic acid lactone, pantolactone, glucoheptonolactone, mannonolactone, galactoheptonolactone; and a combination of two or more thereof.

3. The personal care composition according to claim 1, further comprising from about 0.1 to about 7 wt. % of an additional polyhydroxy acid and/or salt thereof.

4. The personal care composition according to claim 3, wherein the additional polyhydroxy acid and/or salt thereof comprises galactose, lactobionic acid, or a combination thereof.

5. The personal care composition according to claim 1, further comprising from about 0.1 to about 7 wt. % of an alpha hydroxy acid and/or salt thereof, wherein the alpha hydroxy acid comprise glycolic acid, citric acid, lactic acid, malic acid, tartaric acid, phytic acid, hydroxycaprylic acid, hydroxycapric acid, a salt thereof, or a combination of two or more thereof.

6. The personal care composition according to claim 1, further comprising from about 0.1 to about 15 wt. % of an emollient, based on the total weight of the personal care composition, wherein the emollient is selected from the group consisting of: isododecane, sodium starch octenylsuccinate, C₁₂₋₁₅ alkyl benzoate, mineral oil, caprylyl glycol, dimethicone, dicaprylyl ether, ethyl trisiloxane, PPG-14 butyl ether, PPG-3 myristyl ether, stearyl alcohol, stearic acid and salts thereof, glyceryl monoricinoleate, isobutyl palmitate, isocetyl stearate, isocetyl stearate, sulphated tallow, oleyl alcohol, propylene glycol, isopropyl laurate, mink oil, sorbitan stearate, cetyl alcohol, hydrogenated castor oil, stearyl stearate, hydrogenated soy glycerides, isopropyl isostearate, hexyl laurate, dimethyl brassylate, decyl oleate, diisopropyl adipate, n-dibutyl sebacate, diisopropyl sebacate, 2-ethyl hexyl palmitate, isononyl isononanoate, isodecyl isononanoate, isotridecyl isononanoate, 2-ethyl hexyl palmitate, 2-ethyl hexyl stearate, Di-(2-ethyl hexyl) adipate), Di-(2-ethyl hexyl) succinate, isopropyl myristate, isopropyl palmitate, isopropyl stearate, octacosanol, butyl stearate, polyethylene glycols, oleic acid, triethylene glycol, lanolin, castor oil, acetylated lanolin alcohols, acetylated lanolin, petrolatum, isopropyl ester of lanolin, fatty acids, mineral oils, butyl myristate, isostearic acid, palmitic acid, PEG-8 distearate, PEG-23 oleyl ether, olelyl oleate, isopropyl linoleate, cetyl lactate, lauryl lactate, myristyl lactate, quaternised hydroxy alkyl, aminogluconate, vegetable oils, isodecyl oleate, isostearyl neopentanoate, myristyl myristate, oleyl ethoxy myristate, diglycol stearate, ethylene glycol monostearate, myristyl stearate, isopropyl lanolate, paraffin waxes, glycyrrhizic acid, hydrocyethyl stearate amide, and a combination of two or more thereof.

7. The personal care composition according to claim 1, further comprising from about 0.1 to about 25 wt. % of a polyol, based on the total weight of the personal care composition, wherein the polyol comprises ethylene glycol, propylene glycol, butylene glycol, hexylene glycol, glycerin, diglycerin, diethylene glycol, and dipropylene glycol, or a combination of two more thereof.

8. The personal care composition according to claim 1, further comprising from about 0.5 to about 9 wt. % of a structuring agent, based on the total weight of the personal care composition.

9. The personal care composition according to claim 1, wherein the personal care composition comprises a thickening agent selected from the group consisting of: xanthan gum, guar gum, biosaccharide gum, cellulose, acacia seneca gum, sclerotium gum, agarose, pectin, gellan gum, hyaluronic acid, and a combination of two or more thereof.

10. The personal care composition according to claim 1, further comprising from about 0.1 to about 7 wt. % of a preservative, based on the total weight of the personal care composition.

11. The personal care composition according to claim 10, wherein the preservative comprises caprylyl glycol, phenoxyethanol, butylated hydroxytoluene, ethylenediaminetetraacetic acid, ethylhexylglycerin, citric acid, benzoic acid, or a combination of two or more thereof.

12. A personal care composition comprising:
an antiperspirant component comprising gluconolactone and/or an acid thereof; and
from about 0.1 to about 7 wt. % of a thickening agent; and
from about 0.1 to about 25 wt. % of a polyol,
wherein all weight percentages are based on the total weight of the personal care composition.

13. A personal care composition comprising:
an antiperspirant component consisting essentially of gluconolactone and/or an acid thereof.

14. The personal care composition according to claim 1, wherein the antiperspirant component consists of gluconolactone and/or an acid thereof.

15. The personal care composition according to claim 1, wherein the personal care composition is essentially free of aluminum salts.

16. The personal care composition according to claim 1, wherein the personal care composition is essentially free of zinc-based antiperspirant actives, iron-based antiperspirant actives, zirconium-based antiperspirant actives, titanium-based antiperspirant actives, and magnesium-based antiperspirant actives.

17. The personal care composition according to claim 1, wherein the antiperspirant component consists essentially of gluconolactone, lactobionic acid, maltobionic acid, galactonolactone, glucuronolactone, galacturonolactone, gulonolactone, ribonolactone, saccharic acid lactone, pantolactone, glucoheptonolactone, mannonolactone, galactoheptonolactone, an acid thereof, a salt thereof, or a combination of two or more thereof.

18. The personal care composition according to claim 1, wherein the antiperspirant component consists of gluconolactone, lactobionic acid, maltobionic acid, galactonolactone, glucuronolactone, galacturonolactone, gulonolactone, ribonolactone, saccharic acid lactone, pantolactone, glucoheptonolactone, mannonolactone, galactoheptonolactone, an acid thereof, a salt thereof, or a combination of two or more thereof.

19. A method for treating hyperhidrosis in a mammalian subject, the method comprising: applying a personal care composition according to claim 1, to a skin surface of a mammalian subject in need thereof.

20. A method for reducing or preventing sweat production in a mammalian subject, the method comprising: applying a personal care composition according to claim 1, to a skin surface of a mammalian subject in need thereof.
